(19) United States
(12) Patent Application Publication
(10) Pub. No.: US 2006/0047436 A1
(43) Pub. Date: Mar. 2, 2006

(54) System and method for magnifying an immune response

(76) Inventors: Muriel Y. Ishikawa, Livermore, CA (US); Edward K.Y. Jung, Bellevue, WA (US); Nathan P. Myhrvold, Medina, WA (US); Richa Wilson, San Francisco, CA (US); Lowell L. Wood JR., Livermore, CA (US)

Correspondence Address: Searete LLC, Suite 110, 1756-114th Ave. S.E., Bellevue, WA, 98004, US

(21) Appl. No.: 10/926,753

(22) Filed: Aug. 25, 2004

(51) Int. Cl. A61K 39/00; G06F 19/00

(52) U.S. Cl. 702/19; 424/184.1

(57) ABSTRACT

The present application relates, in general, to a system and/or method for detection and/or treatment.

### CROSS-REFERENCE TO RELATED APPLICATIONS

The present application is related to, claims the earliest available effective filing date(s) from (e.g., claims earliest available priority dates for other than provisional patent applications; claims benefits under 35 USC § 119(e) for provisional patent applications), and incorporates by reference in its entirety all subject matter of the following listed applications; the present application also claims the earliest available effective filing date(s) from, and also incorporates by reference in its entirety all subject matter of any and all parent, grandparent, great-grandparent, etc. applications of the following listed applications:
1. U.S. patent application entitled A SYSTEM AND METHOD RELATED TO IMPROVING AN IMMUNE SYSTEM naming Muriel Y. Ishikawa, Edward K. Y. Jung, Nathan P. Myhrvold, Richa Wilson, and Lowell L. Wood, Jr. as inventors, filed 24 Aug. 2004.
2. U.S. patent application entitled A SYSTEM AND METHOD RELATED TO ENHANCING AN IMMUNE SYSTEM naming Muriel Y. Ishikawa, Edward K. Y. Jung, Nathan P. Myhrvold, Richa Wilson, and Lowell L. Wood, Jr. as inventors, filed 24 Aug. 2004.
3. U.S. patent application entitled A SYSTEM AND METHOD RELATED TO AUGMENTING AN IMMUNE SYSTEM naming Muriel Y. Ishikawa, Edward K. Y. Jung, Nathan P. Myhrvold, Richa Wilson, and Lowell L. Wood, Jr. as inventors, filed 24 Aug. 2004.
4. U.S. patent application entitled A SYSTEM AND METHOD FOR HEIGHTENING AN IMMUNE RESPONSE naming Muriel Y. Ishikawa, Edward K. Y. Jung, Nathan P. Myhrvold, Richa Wilson, and Lowell L. Wood, Jr. as inventors, filed contemporaneously herewith.

### TECHNICAL FIELD

The present application relates, in general, to detection and/or treatment.

### SUMMARY

In one aspect, a method includes but is not limited to: delivering a treatment to a host wherein the treatment is associated with modulating a predicted pattern of progression of one or more antigenic determinants. In addition to the foregoing, other method aspects are described in the claims, drawings, and text forming a part of the present application.

In another aspect, a system includes but is not limited to: circuitry for suggesting delivery of a treatment to a host wherein the treatment is associated with modulating a predicted pattern of progression of one or more antigenic determinants. In addition to the foregoing, other system aspects are described in the claims, drawings, and text forming a part of the present application.

In another aspect, a system includes but is not limited to: a computer readable medium including, but not limited to, a computer program for use with a computer system and wherein the computer program includes a plurality of instructions including: (a) one or more instructions for selecting one or more determining elements; (b) one or more instructions for predicting at least one pattern change in the one or more determining elements; (c) one or more instructions for associating the at least one pattern change in the one or more determining elements with at least one outcome; and (d) one or more instructions for designating a course of action associated with the at least one pattern change in the one or more determining elements. In addition to the foregoing, other system aspects are described in the claims, drawings, and text forming a part of the present application.

In another aspect, a system includes but is not limited to: circuitry for selecting one or more determining elements; circuitry for predicting at least one pattern change in the one or more determining elements; circuitry for associating the at least one pattern change in the one or more determining elements with at least one outcome; and circuitry for designating a course of action associated with the at least one pattern change in the one or more determining elements. In addition to the foregoing, other system aspects are described in the claims, drawings, and text forming a part of the present application.

In one or more various aspects, related systems include but are not limited to circuitry and/or programming for effecting the herein-referenced method aspects; the circuitry and/or programming can be virtually any combination of hardware, software, and/or firmware configured to effect the herein- referenced method aspects depending upon the design choices of the system designer.

In addition to the foregoing, various other method and or system aspects are set forth and described in the text (e.g., claims and/or detailed description) and/or drawings of the present application.

The foregoing is a summary and thus contains, by necessity; simplifications, generalizations and omissions of detail; consequently, those skilled in the art will appreciate that the summary is illustrative only and is NOT intended to be in any way limiting. Other aspects, inventive features, and advantages of the devices and/or processes described herein, as defined solely by the claims, will become apparent in the non-limiting detailed description set forth herein.

### BRIEF DESCRIPTION OF THE FIGURES

FIG. 1 is a diagrammatic view of one aspect of an exemplary interaction of an immune response component, for example, an antibody interacting with an epitope displayed by an agent.

FIG. 2 is a diagrammatic view of one aspect of a method of enhancing an immune system.

FIG. 3 depicts one aspect of an antigen antibody interaction showing the occurrence of mutational changes in a selected epitope and corresponding changes in a complementary antibody.

FIG. 4 is an illustration of one aspect of mutational changes in an epitope displayed by an agent and the corresponding changes in an immune response component, for example, an antibody.

FIG. 5 depicts a high-level logic flow chart of a process.

FIG. 6 depicts a high-level logic flowchart depicting alternate implementations of the high-level logic flowchart of FIG. 5.

FIG. 7 depicts a high-level logic flowchart depicting alternate implementations of the high-level logic flowchart of FIG. 5.

FIG. 8 depicts a high-level logic flowchart depicting alternate implementations of the high-level logic flowchart of FIG. 5.

FIG. 9 depicts a high-level logic flowchart depicting alternate implementations of the high-level logic flowchart of FIG. 5.

FIG. 10 depicts a high-level logic flowchart depicting alternate implementations of the high-level logic flowchart of FIG. 5.

FIG. 11 depicts a high-level logic flowchart depicting alternate implementations of the high-level logic flowchart of FIG. 5.

FIG. 12 depicts a high-level logic flowchart depicting alternate implementations of the high-level logic flowchart of FIG. 5.

FIG. 13 depicts a high-level logic flowchart depicting alternate implementations of the high-level logic flowchart of FIG. 5.

FIG. 14 depicts a partial view of a system that may serve as an illustrative environment of and/or for subject matter technologies.

FIG. 15 depicts a partial view of a system that may serve as an illustrative environment of and/or for subject matter technologies.

The use of the same symbols in different drawings typically indicates similar or identical items.

### DETAILED DESCRIPTION

The present application uses formal outline headings for clarity of presentation. However, it is to be understood that the outline headings are for presentation purposes, and that different types of subject matter may be discussed throughout the application (e.g., device(s)/structure(s) may be described under the process(es)/operations heading(s) and/or process(es)/operations may be discussed under structure(s)/process(es) headings). Hence, the use of the formal outline headings is not intended to be in any way limiting.

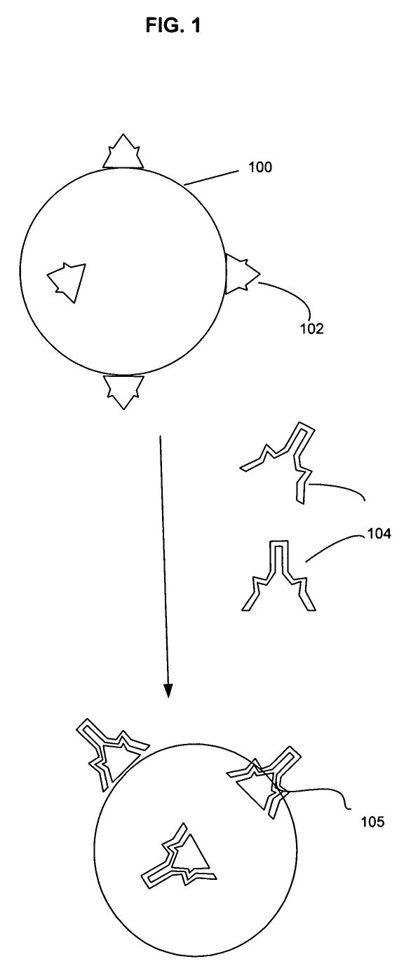

With reference now to FIG. 1, depicted is a diagrammatic view of one aspect of an exemplary interaction of an immune response component, for example, an antibody interacting with an epitope displayed by an agent. Accordingly, the present application first describes certain specific exemplary methods of FIG. 1; thereafter, the present application illustrates certain specific exemplary structures. Those having skill in the art will appreciate that the specific structures and processes described herein are intended as merely illustrative of their more general counterparts. It will also be appreciated by those of skill in the art that an antigen-antibody interaction is an exemplary interaction of the interaction of an immune response component with an antigen. Therefore, although, the exact nature of the interaction may vary the overall picture as described herein and in other related applications relates to the interaction of an immune response component interacting with the antigen.

A. Structure(s) and or System(s)

With reference to the figures, and with reference now to FIG. 1, depicted is a diagrammatic view of one aspect of an exemplary interaction of an immune response component, for example, an antibody 104 interacting with an epitope 102 displayed by an agent 100.

The term “agent” 100 may include, for example, but is not limited to, an organism, a virus, a bacterium, a yeast, a mold, a fungus, a mycoplasma, an ureaplasma, a Chlamydia, a rickettsia, a nanobacterium, a prion, an agent responsible for a transmissible spongiform encephalopathy (TSE), a multicellular parasite, a protein, an infectious protein, a nucleic acid, a metabolic by product, a cellular by product, and/or a toxin. The term “agent” 100 may include, but is not limited to, a putative causative agent of a disease or disorder, a cell that is deemed, for example, a target for therapy, a target for neutralization, and/or or a cell whose removal may prove beneficial to the host. The term “agent” 100 may also include, but is not limited to, a byproduct of a cell that may be neutralized and/or whose removal may prove beneficial to the host. Furthermore, the term “agent” 100 may include an agent belonging to the same family or a group, or an agent exhibiting a common and/or a biological function.

The term “antibody” 104 as used herein, is used in the broadest possible sense and may include but is not limited to an antibody, a recombinant antibody, a genetically engineered antibody, a chimeric antibody, a monospecific antibody, a bispecific antibody, a multispecific antibody, a diabody, a chimeric antibody, a humanized antibody, a human antibody, a heteroantibody, a monoclonal antibody, a polyclonal antibody, and/or an antibody fragment. The term “antibody” may also include but is not limited to types of antibodies such as IgA, IgD, IgE, IgG and/or IgM, and/or the subtypes IgG1, IgG2, IgG3, IgG4, IgA1 and/or IgA2. The term antibody may also include but is not limited to an antibody fragments such as at least a portion of an intact antibody 104, for instance, the antigen binding variable region. Examples of antibody fragments include Fv, Fab, Fab′, F(ab′), F(ab′).sub.2, Fv fragments, diabodies, linear antibodies single-chain antibody molecules, multispecific antibodies, or other antigen binding sequences of antibodies. Additional information may be found in U.S. Pat. No. 5,641,870, U.S. Pat. No. 4,816,567, WO 93/11161, Holliger Et Al., Diabodies: Small Bivalent And Bispecific Antibody Fragments, PNAS (Proc. Natl. Acad. Sci. USA), 90: 6444-6448 (1993), Zapata et al., Engineering Linear F(Ab′)2 Fragments For Efficient Production In Escherichia Coli And Enhanced Antiproliferative Activity, Protein Eng. 8(10): 1057-1062 (1995), which are incorporated herein by reference. Antibodies may be generated for therapeutic purposes by a variety of known techniques, such as, for example, phage display, and/or transgenic animals.

The term “heteroantibodies”, as used herein, may include but is not limited to, two or more antibodies, antibody fragments, antibody derivatives, and/or antibodies with at least one specificity linked together. Additional information may be found in U.S. Pat. No. 6,071,517, which is incorporated herein by reference.

The term “chimeric antibodies”, as used herein, may include but is not limited to antibodies having mouse variable regions joined to human constant regions. In one aspect, “chimeric antibodies” includes antibodies with human framework regions combined with complementarity determining regions (CDR's) obtained from a mouse and/or rat; those skilled in the art will appreciate that CDR's may be obtained from other sources. Additional information may be found in EPO Publication No 0239400, which is incorporated herein by reference. Although the foregoing has referred to the plural term “chimeric antibodies,” those having skill in the art will appreciate that the singular term “chimeric antibody” may include but is not limited to singular instances of examples given for the plural term, as appropriate to context (see, e.g., the as-filed claims). The same is generally true for the use of substantially any plural and/or singular terms as used herein; that is, those having skill in the art can translate from the plural to the singular and/or from the singular to the plural as is appropriate to the context or application, and hence the various singular/plural permutations are not expressly set forth herein for sake of clarity.

The term “humanized antibody”, as used herein, may include but is not limited to an antibody having one or more human regions, and/or a chimeric antibody with one or more human regions, also considered the recipient antibody, combined with CDR's from a donor mouse and/or rat immunoglobulin. In one aspect, humanized antibodies may include residues not found in either donor or recipient sequences. Humanized antibodies may have single and/or multiple specificities. Additional information may be found in U.S. Pat. No. 5,530,101, and U.S. Pat. No. 4,816,567, which are incorporated herein by reference. Information may also be found in, Jones et al., Replacing The Complementarity-Determining Regions In A Human Antibody With Those From A Mouse, Nature, 321:522-525(1986); Riechmann et al., Reshaping Human Antibodies For Therapy, Nature, 332:323-327 (1988); and Verhoeyen et al., Reshaping Human Antibodies: Grafting An Antilysozyme Activity, Science, 239:1534 (1988), which are all incorporated herein by reference.

The term “human antibodies”, as used herein, may include but is not limited to antibodies with variable and constant regions derived from human germline immunoglobulin sequences. The term human antibodies may include is not limited to amino acid residues of non-human origin, encoded by non-human germline, such as, for example, residues introduced by site directed mutations, random mutations, and/or insertions. Methods for producing human antibodies are known in the art and incorporated herein by reference. Additional information may be found in U.S. Pat. No. 4,634,666, which is incorporated herein by reference.

The term “recombinant antibody”, as used herein, may include antibodies formed and/or created by recombinant technology, including, but not limited to, chimeric, human, humanized, hetero antibodies and the like.

The term “immune response component”, as used herein, may include, but is not limited to, at least a part of a macrophage, a lymphocyte, a T-lymphocyte, a killer T-lymphocyte, an immune response modulator, a helper T-lymphocyte, an antigen receptor, an antigen presenting cell, a cytotoxic T-lymphocyte, a T-8 lymphocyte, a CD1 molecule, a B lymphocyte, an antibody, a recombinant antibody, a genetically engineered antibody, a chimeric antibody, a monospecific antibody, a bispecific antibody, a multispecific antibody, a diabody, a chimeric antibody, a humanized antibody, a human antibody, a heteroantibody, a monoclonal antibody, a polyclonal antibody, an antibody fragment, and/or synthetic antibody.

Continuing to refer to FIG. 1, the epitope 102 or parts thereof may be displayed by the agent 100, may be displayed on the surface of the agent 100, extend from the surface of the agent 100, and/or may only be partially accessible by the immune response component. The term “epitope” 102, as used herein, may include, but is not limited to, a sequence of at least 3 amino acids, a sequence of at least nine nucleotides, an amino acid residue, a nucleotide, a carbohydrate, a protein, a lipid, a capsid protein, a polysaccharide, a lipopolysaccharide, a glycolipid, a glycoprotein, and/or or at least a part of a cell. As used herein, the term “epitope” 102 may be used interchangeably with antigen, paratope binding site, antigenic determinant, and/or determinant. As used herein, the term determinant can include an influencing or determining element or factor, unless context indicates otherwise. In one aspect the term “epitope” 102 includes, but is not limited to, a peptide binding site. As used herein, the term “epitope” 102 may include structural and/or functionally similar sequences found in the agent 100. The term “epitope” 102 includes, but is not limited to, similar sequences observed in orthologs, paralogs, homologs, isofunctional homologs, heterofunctional homologs, heterospecific homologs, and/or pseudogenes of the agent 100.

In one aspect, the epitope 102 may be a linear determinant. For example, the sequences may be adjacent to each other. In another aspect, the epitope 102 is a non-linear determinant, for example, including juxtaposed groups which are non-adjacent but become adjacent to each other on protein folding. Furthermore, the sequence of the non-linear determinant may be derived by proteasomal processing and/or other mechanisms and the sequence synthetically made for presentation to the immune response component.

Continuing to refer to FIG. 1, in one aspect, the immune system launches a humoral response producing antibodies capable of recognizing and/or binding to the epitope 102 followed by the subsequent lysis of the agent 100. Mechanisms by which the antigen 102 elicits an immune response are known in the art and such mechanisms are incorporated herein by reference. In one aspect, the binding of the antibody 104 to the epitope 102 to form an antigen-antibody complex 105 is characterized as a lock and key fit.

The epitope 102 may include any portion of the agent. In one aspect, the epitope 102 may include at least a portion of a gene. In another aspect the epitope may include at least a part of a non-coding region.

In one aspect, the epitope 102 is capable of evoking an immune response. The strength and/or type of the immune response may vary, for example, the epitope 102 may invoke a weak response and/or a medium response as measured by the strength of the immune response. It is contemplated that in one instance the epitope 102 selected for targeting may be one that invokes a weak response in the host, however, it may be selective to the agent 100. In another example, the epitope 102 selected may invoke a weak response in the host, however it may be selected for targeting as it is common to agents deemed as targets. The herein described implementations are merely exemplary and should be considered illustrative of like and/or more general implementations within the ambit of those having skill in the art in light of the teachings herein.

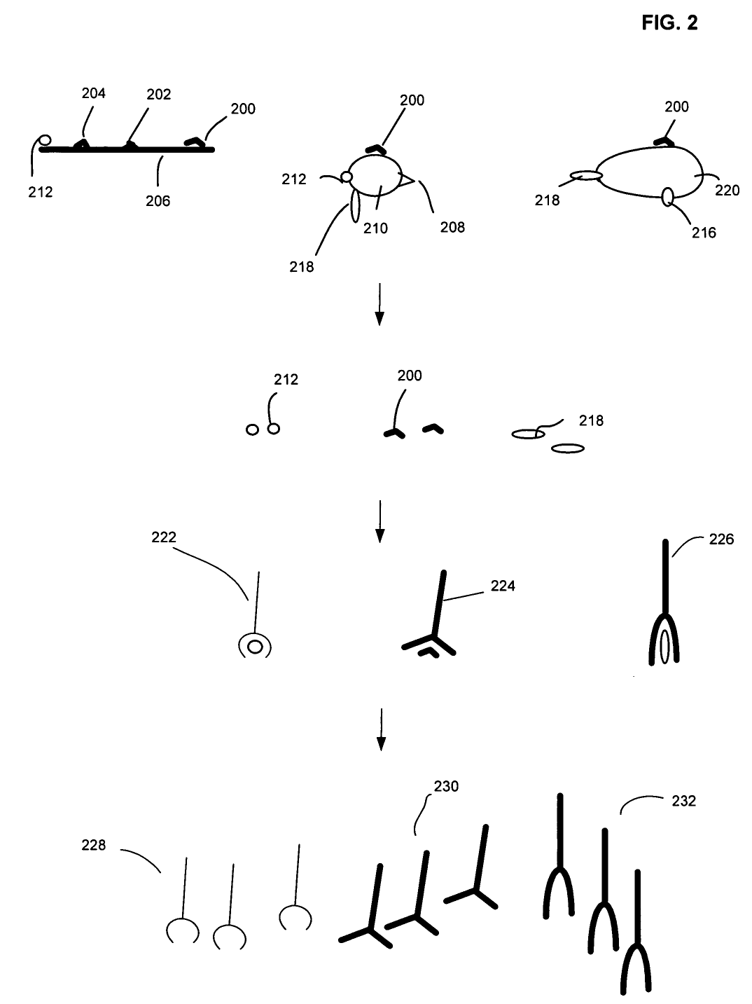

With reference to the figures, and with reference now to FIG. 2 depicted is a diagrammatic view of one aspect of a method of enhancing an immune system. In one aspect, an effective treatment therapy towards a disease and/or a disorder may utilize one or more immune response components designed to recognize one or more antigens common to one or more agents. Such common antigens may represent an effective target group of antigens. The immune response components designed to seek out and neutralize the common antigens may be effective against one or more agents.

With reference now to FIGS. 1 and 2, in one aspect, a shared epitope 200 is depicted as common to three agents 206, 210 and 220. In another aspect, a second shared epitope 212 is common to two agents 206 and 210. In yet another aspect, a third shared epitope 218 is common to two agents 210 and 220. Finding a subset of common epitopes shared amongst one or more agents may be done by statistical analysis, for example, by metaprofiling. One variation of this aspect is identification of at least one common epitope shared with one or more agents also referred to as an antigenic profile, and/or an antigenic signature. Additional information may be found in a publication by Rhodes et al., Large Scale Meta-Analysis Of Cancer Micorarray Data Identifies Common Transcriptional Profiles Of Neoplastic Transformation And Progression, PNAS Jun. 22, 2004, 101:(25) 9309-9314, and is incorporated herein by reference.

Continuing to refer to FIGS. 1 and 2, in one aspect, one or more agents 206, 210, and 220 depicted may share a subset of common epitopes. The selection of epitopes may depend on a number of criteria. For example, the initial selection may be based on, including, but not limited to, the number of instances of occurrences of the epitope 102 by one or more agents, the number of instances of occurrence of the epitope 102 by the agent 100, the location of the epitope 102, the size of the epitope 102, the nature of the epitope 102, the sequence identity and/or homology of the epitope 102 with host sequences, the composition of the epitope 102, and/or putative known or predicted changes in the epitope 102 sequence. The selection of epitopes may also depend on, for example, the type of immune response component desired for treating and/or managing the disease, disorder, and/or condition.

In one aspect, the epitope 102 selected has a probable sequence match with an entity. The term “entity”, as used herein, may include the agent 100 and/or a host depending on context. For example, whether the term entity includes the agent 100, the host, or both will sometimes depend, for example, on the nature of a described interaction. The term“host”, as used herein, may include but is not limited to an individual, a person, a patient, and/or virtually anyone requiring management of a disease, disorder, and/or condition. For example, the epitope 102 selected may have a 0-70% sequence match at the amino acid level with the entity, for example, the host, or a 0-100% sequence match with the entity, for example, the host or the agent 100. Those having skill in the art will recognize that part of that context in relation to the term “entity” is that generally what is desired is a practicably close sequence match to the agent (e.g., HIV), so that the one or more immune system components in use can attack it and a practicably distant sequence match to the host (e.g., a patient), in order to decrease or render less aggressive any attack by the immune system components in use on the host. However, it is also to be understood that in some contexts the agent will in fact constitute a part of the host (e.g., when the agent to be eradicated is actually a malfunctioning part of the host, such as in an auto-immune disease), in which case that part of the host to be eradicated will be treated as the “agent”, and that part of the host to be left relatively undisturbed will be treated as the “host.” In another aspect, the epitope 102 selected has a sequence match with the entity, for example, a high sequence match, a relatively higher sequence match with other agents compared to the host, or a 0-100% sequence match with the agent 100. The term “sequence match”, as used herein, includes both sequence matching at the nucleic acid level and/or at the protein level. In an embodiment, the epitope 102 selected has a low probable sequence match with the host. In another embodiment, the epitope 102 selected has a high sequence match with other agents.

In another aspect the epitope 102 selected has a likely and/or a probable sequence match with other epitopes, for example, including, but not limited to, the epitope 102 having a structural sequence match, a functional sequence match, a similar functional effect, a similar result in an assay and/or a combination. Structural comparison algorithms and/or 3-dimensional protein structure data may be used to determine whether two proteins may have a structural sequence match. In another example the epitope 102 may have a functional match and/or share a similar functional effect with epitopes of interest. In this example, the epitope 102 may have a lower probable sequence match but may still exert the same functional effect. In another example, the epitope 102 and/or other epitopes of interest may have a lower probable sequence match but may share similar activities, for example, enzymatic activity and/or receptor binding activity, as determined by using an assay.

In another aspect the epitope 102 selected may be an immunological effective determinant, for example, the epitope 102 may be weakly antigenic, however it may invoke an effective immune response relating to, for example, the nature and/or the type of the immune response component it invokes. In another aspect the epitope 102 may exert a similar effect on the immune response, for example, the epitope 102 selected may be part of the antigenic structure of an agent unrelated to the disease or disorder in question, however, it may exert a substantially similar effect on the immune system as measured by, for example, the type, the nature, or the period of the immune response.

In one aspect, a sequence match with an entity may be determined by, for example, calculating the percent identity and/or percent similarity between epitopes and/or between the epitope 100 and the host. In one aspect, the percent identity between two sequences may be calculated by determining a number of substantially similar positions obtained after aligning the sequences and introducing gaps. For example, in one implementation the percent identity between two sequences is treated as equal to (=) a number of substantially similar positions/total number of positions ×100. In this example, the number and length of gaps introduced to obtain optimal alignment of the sequences is considered. In another aspect, the percent identity between two sequences at the nucleic acid level may be determined by using a publicly available software tool such as BLAST, BLAST-2, ALIGN and/or DNASTAR software. Similarly, the percent identity between two sequences at the amino acid level may be calculating by using publicly available software tools such as, for example, Peptidecutter, AACompSim, Find Mod, GlycoMod, InterProtScan, DALI and/or tools listed on the ExPasy (Expert Protein Analysis System) Proteomics Server at http://www.expasy.org/. In one embodiment, the percent identity at the nucleic acid level and at the amino acid level are determined.

It will be appreciated by those skilled in the art that the epitope 102 selected need not be limited to a matching sequence displayed by the agent 100. In one aspect, a meta signature and/or a consensus sequence may be derived based on any number of criteria. In one aspect, the meta signature may be derived by analysis of data from sources such as, for example, antigenic evolution, genetic evolution, antigenic shift, antigenic drift, data from crystal structure, probable match with a host, probable match with other strains, and/or strength of the immunogenic response desired. The meta signature may include new sequences and/or may exclude some sequences. For example, it may include silent mutations, mismatches, a spacer to bypass a hotspot or a highly mutagenic site, predicted changes in the sequence, and/or may include epitopes from multiple agents thus providing protection from multiple agents. As another example, the meta signature may exclude sequences, such as, for example, including, but not limited to, mutagenic sequences and/or sequences with a match to the host.

In one aspect, the predicted changes in the epitope 102 may be determined by analysis of past variations observed and/or predicted in the agent 100 (e.g., FIG. 1). Computational analysis can be used to determine regions showing sequence variations and/or hot spots. In one aspect, high speed serial passaging may be performed computationally mimicking the serial passaging that occurs naturally with a production of a new strain of the agent 100. It will be appreciated by those of skill in the art that the hot spots need not be identified by examining the epitope 102, and/or by examining the epitope 102 in context with the agent 100. Information pertaining to hot spots can also be extrapolated by performing sequence analysis of other agents and/or domain analysis of the other agents. For example, in one implementation the epitope 102 may be part of a domain shared between multiple agents which may lack the epitope 102 of interest. Information pertaining to hotspots identified in the domain of the other agents may be of practical use in determining the metasignature.

In one aspect, one or more sets and/or subsets of epitopes may be formed. The nature and type of criteria used to form the sets and/or subsets will depend, for example, on the nature and type of the agent 100, the duration of the immune response desired (e.g., short-term immunity, or long-term immunity), the nature of the immune response desired (e.g., weak, moderate, or strong), the population seeking protection (e.g., presence of prior exposure) and the like. The sets and subsets so formed may accept input either robotically or from a user (e.g., a manufacturer of immune response components, wet lab, or medical personnel).

The pattern changes predicted in the epitope 102 may be supplemented, for example, by other methodology, statistical analysis, historical data, and other extrapolations of the type utilized by those having skill in the art. The knowledge of these predicted pattern changes represents an arsenal in the design and/or selection of the immune response components. The predicted pattern changes may be used to determine the progression of the changes in the immune response component required to manage such changes. Inferring the pattern changes in the epitope 102 and using the information to modulate the progressing response may help manage the response more effectively. For example, the pattern changes may be used to provide a timeline of when the therapy could be changed, what therapy should constitute the change, or the duration of the change. As a more specific example, one reason why Human Immunodeficiency Virus (HIV) is able to successfully kill its host is that the virus mutates faster than the immune system can track and respond to its mutations. In a specific implementation of the subject matter described herein, a sample of HIV is taken from a patient at a point in time and computational biological techniques are used to infer likely mutations of the virus at future times. Cloning techniques are then utilized to synthesize immune system activating aspects of the future HIV strains, and thereafter subsequent cloning techniques are utilized to rapidly generate copious amounts of one or more immune system components (e.g., antibodies) that are keyed to the likely future generation of the patient's HIV. Once cloned, the immune system components are then loaded back to the patient and thus are present and waiting for the HIV when it mutates. If the HIV mutates as anticipated, the preloaded immune response components attack the mutated HIV, thereby likely greatly reducing the presence of the HIV. In another implementation, the actual mutation of the HIV is manually tracked, and once the actual mutation has been determined, yet more cloning techniques are utilized to generate yet more immune system components appropriate to the mutated virus.

In one aspect, the epitope 102 selected for designating the immune response component may be synthetically made and/or derived from the agent 100. In one embodiment the epitope 102 selected is derived from an agent 100 extracted from an individual desiring treatment and/or an individual found resistant to that agent. In one aspect the epitope 102 selected for designating the immune response component may include multiple copies of the exact same epitope and/or multiple copies of different epitopes.

In one aspect the metasignature includes sequences matching adjacent and/or contiguous sequences. In another aspect the metasignature includes non adjacent sequences. For example, it will be appreciated by those of skill in the art that peptide splicing and/or proteosomal processing of the epitope 102 that occurs naturally may result in the formation of a new epitope, for example, a non-linear epitope. In this example, proteosomal processing may result in the excision of sequences transposing non-contiguous sequences to form the non-linear epitope. Additional information may be found in Hanada et al., Immune Recognition Of A Human Renal Cancer Antigen Through Post-Translational Protein Splicing, Nature 427:252 2004), and Vigneron et al., An Antigenic Peptide Produced By Peptide Splicing In The Proteasome, Science 304:587 (2004) hereby incorporated by reference herein in its entirety.

Additionally, it will also be appreciated by those of skill in the art that the metasignature may include sequences displayed on two different parts of the agent 100. For example, non adjacent sequences may appear adjacent each other when the protein is folded. In this aspect, the metasignature may include the nonadjacent sequences for identifying the metasignature. Furthermore, the metasignature may include nonadjacent sequences corresponding to a specific conformational state of a protein. Immune response components designed to bind such sequences may be specific to the conformational state of the protein. 3-D and/or crystal structure information may also be used to designate the metasignature.

In one aspect, the metasignature may include multiple sets of epitopes targeting a predicted pattern change and/or an observed pattern change. For example, multiple sets of epitopes may be designed for vaccination and/or for production of immune response components.

Techniques for epitope mapping are known in the art and herein incorporated by reference. For example, FACS analysis and ELISA may be used to investigate the binding of antibodies to synthetic peptides including at least a portion of the epitope. Epitope mapping analysis techniques, Scatchard analysis and the like may be used to predict the ability of the antibody 104 to bind to the epitope 102 presented on the agent 100, to determine the binding affinity of the antibody 104 to the epitope 102, and/or to discern a desirable configuration for the antibody 104.

Continuing to refer to FIG. 2, in one aspect, for example, the sequences of selected epitopes 200, 212, and 218 may be used to design one or more complementary antibodies 224, 222, and 226, respectively. Techniques for making antibodies are known in the art and are incorporated herein by reference. The purified complementary antibodies 230, 228, or 232 may then be made available for therapeutic and/or prophylactic treatment.

The term “an effective treatment therapy”, as used herein, includes, but is not limited to, the use of immune response components in combination with other antibodies, antibody fragments, and/or in combination with other treatments, including, but not limited to, drugs, vitamins, hormones, medicinal agents, pharmaceutical compositions and/or other therapeutic and/or prophylactic combinations. In another aspect, the immune response component may be used in combination, for example, with a modulator of an immune response and/or a modulator of an antibody. In one aspect, cocktails of immune response components may be administered, for example, by injecting by a sub-cutaneous, nasal, intranasal, intramuscular, intravenous, intraarterial, intrathecal, intracapsular, intraorbital, intracardiac, transdermal, intradermal, intraperitoneal, transtracheal, subcuticular, intraarticular, subcapsular, subarachnoidal, intraspinal, epidural, intrastemal, infusion, topical, sublingual, and/or enteric route.

The therapeutic effect of the immune response component may be produced by one or more modes of action. For example, in one aspect, the immune response component may produce a therapeutic effect and/or alleviate the symptoms by targeting specific cells and neutralizing them. In another aspect, the immune response component may bind to and/or block receptors present on the agent 100 and/or may directly and/or indirectly block the binding of molecules, such as, for example, cytokines, and/or growth factors, to the agent 100. In another aspect, the therapeutic effect of the immune response component is produced by functioning as signaling molecules. In this example, the immune response component may induce cross linking of receptors with subsequent induction of programmed cell death.

The immune response component may be engineered to include, for example, one or more effector molecules, such as, for example, drugs, small molecules, enzymes, toxins, radionuclides, cytokines, and/or DNA molecules. In this example, the immune response component may serve as a vehicle for targeting and binding the agent 100 and/or delivering the one or more effector molecules. In one aspect, the immune response component may be engineered to include the one or more effector molecules without the natural effector functions of the immune response component.

In another aspect, one or more immune response components may be coupled to molecules for promoting immune system cells to eliminate unwanted cells. This technique has been described for the treatment of tumors, viral infected cells, fungi, and bacteria using antibodies. Additional information may be found in U.S. Pat. No. 4,676,980 to Segal, which is incorporated herein by reference.

The criteria for selection of the one or more immune response components may vary, for example, one criterion may include the strength of the interaction or the binding affinity of the immune response component for the antigen 102. Numerous techniques exist for enhancing the binding affinity of the antibody for the antigen 102. In one aspect, the binding affinity of the antibody for the antigen 102 may be enhanced by constructing phage display libraries from an individual who has been immunized with the antigen 102 either by happenstance or by immunization. The generation and selection of higher affinity antibodies may also be improved, for example, by mimicking somatic hypermutagenesis, complementarity-determining region (CDR) walking mutagenesis, antibody chain shuffling, and/or technologies such as Xenomax technology (available from Abgenix, Inc. currently having corporate headquarters in Fremont, Calif. 94555). In one example, antibodies including introduced mutations may be displayed on the surface of filamentous bacteriophage. Processes mimicking the primary and/or secondary immune response may then be used to select the desired antibodies, for example, antibodies displaying a higher binding affinity for the antigen and/or by the evaluating the kinetics of dissociation. For additional information see, Low et al., Mimicking Somatic Hypermutation: Affinity Maturation Of Antibodies Displayed On Bacteriophage Using A Bacterial Mutator Strain, J. Mol. Biol. 260:359-368 (1996); Hawkins et al. Selection Of Phage Antibodies By Binding Affinity. Mimicking Affinity Maturation, J. Mol. Biol. 226:889-896 (1992), which are incorporated herein by reference.

In another example, the generation and/or selection of higher affinity antibodies may be carried out by CDR walking mutagenesis, which mimics the tertiary immune selection process. For example, saturation mutagenesis of the CDR's of the antibody 104 may be used to generate one or more libraries of antibody fragments which are displayed on the surface of filamentous bacteriophage followed by the subsequent selection of the relevant antibody using immobilized antigen. Sequential and parallel optimization strategies may be used to then select the higher affinity antibody. For additional information see Yang et al., CDR Walking Mutagenesis For The Affinity Maturation Of A Potent Human Anti-HIV-1 Antibody Into The Picomolar Range, J. Mol. Biol 254(3):392-403 (1995), which is incorporated herein by reference in its entirety.

In yet another example, site directed mutagenesis may be used to generate and select higher affinity antibodies, for example, by parsimonious mutagenesis. In this example, a computer based method is used to identify and screen amino acids included in the one or more CDR's of a variable region of an antibody 104 involved in an antigen-antibody binding. Additionally, in some implementations, the number of codons introduced is such that about 50% of the codons in the degenerate position are wildtype. In another example, antibody chain shuffling may be used to generate and select higher affinity antibodies. These techniques are known in the art and are herein incorporated by reference.

The dosage of the immune response component may vary and in one aspect may depend, for example, on the duration of the treatment, body mass, severity of the disease, and/or age. Compositions including immune response components may be delivered to an individual for prophylactic and/or therapeutic treatments. In one aspect, an individual having a disease and/or condition is administered a treatment dose to alleviate and/or at least partially cure the symptoms. In this example, a therapeutically effective dose is administered to the patient.

In another aspect, an individual's resistance may be enhanced by providing a prophylactically measured dose. For example, including, but not limited to, the individual may be genetically vulnerable to the disease and/or condition, the individual may visit a location where the agent 100 is prevalent, or the individual may fear exposure to the agents and/or related agents associated with the disease and/or condition.

Optimization of the physico-chemical properties of the immune response component may be improved, for example, by computer based screening methods. Properties affecting antibody therapeutics may be improved, such as, for example, stability, antigen binding affinity, and/or solubility. Additional information may be found in U.S. patent application Ser. No. 20040110226 to Lazar, which is incorporated herein by reference.

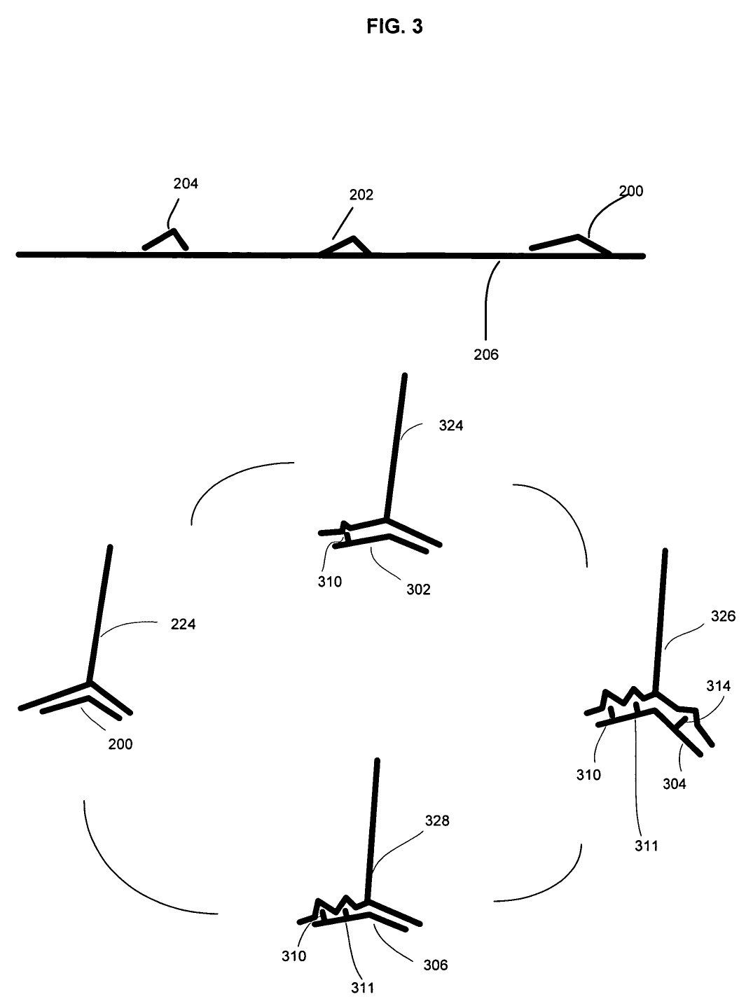

With reference to the figures, and with reference now to FIGS. 1, 2, and 3, depicted is one aspect of the antigen antibody interaction 105 showing the occurrence of mutational changes in the selected epitope 200 and corresponding changes in the complementary antibody 224. Such mutational changes in the epitope 200, for example, may be minor or major in nature. These minor and/or major antigenic variations may render an existing treatment less effective. Thus an effective treatment therapy towards a disease or disorder may include treating the disease or disorder with one or more antibodies designed to anticipate one or more antigenic variations common to one or more agents 100 or one or more related agents. Furthermore, predicting the course of the minor and/or major antigenic variations of the agent 100 and/or the related agents would also be beneficial in designing or selecting the one or more antibodies. Additionally, in some implementations the inclusion of information from SNP databases is helpful in designing antibodies for binding the selected epitope 200.

Minor changes in the epitope 102 which do not always lead to the formation of a new subtype may be caused, for example, by point mutations in the selected epitope 200. In one aspect, the occurrence of point mutations may be localized, for example, to hotspots of the selected epitope 200. The frequency and/or occurrence of such hotspots may be provided by the computer based method. Additionally, the method provides for access to databases including, for example, historical lists of the antigenic variations of the agent 100 and/or of the selected epitope 200, for example, from previous endemics and/or pandemics. Such information may be part of an epitope profile for charting the progression of the immune response. For example, including, but not limited to, a point mutation in the glutamic acid at position 92 of the NS1 protein of the influenza virus has been shown to dramatically downregulate activation of cytokines. Such information may be useful in designating the metasiganture.

Continuing to refer to FIGS. 1, 2, and 3, depicted is that a mutation 310 in the selected epitope 200 results in a mutated epitope 302. The term “the selected epitope 200” as typically used herein, often constitutes a type of the more general term of presented epitope, unless context indicates otherswise. The generation of the mutated epitope 302 may reduce the binding of the immune response component, for example, the antibody 224. In one aspect, effective binding could be enhanced by generating a new antibody 324 corresponding to the mutated epitope 302. The frequency of minor antigenic variations may be predicted by examining known and/or predicted hotspots. For example, additional mutations 311 and/or 314 may be predicted by the computer based method and corresponding antibodies 328 and/or 326 respectively, designed to factor such antigenic variations in the mutated epitopes 306 and/or 304, respectively. In one aspect, an effective treatment therapy, may incorporate this knowledge in providing an effective humoral response towards an agent 100. For example, a cocktail of immune response components may include the antibodies 224, 324, 326, 328 for binding to the selected epitope 200 and/or its predicted mutated versions. In one aspect, the cocktail of one or more antibodies may be supplemented by additional chemicals, growth factors, drugs, or growth factors. In another aspect, the effective treatment therapy may include varying doses of inmmune response components, for example, a substantially larger dosage of 326 relative to 324, 328, and/or 224.

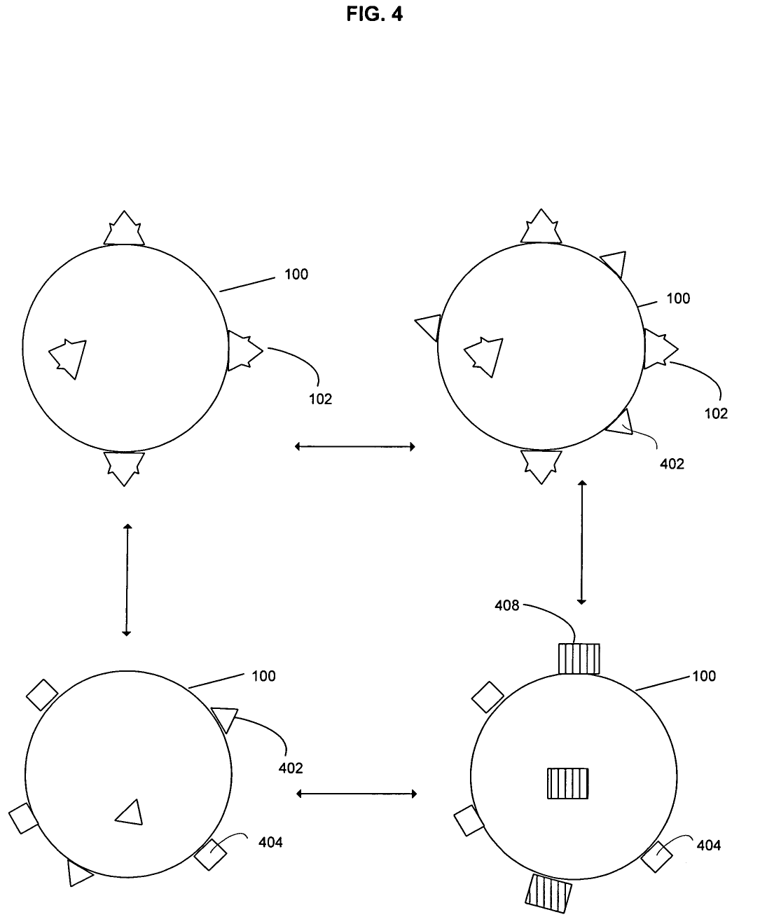

Referring now to FIG. 4, for example, one or more new epitopes 402, 404, 406, and/or 408 may appear on the surface of the agent 100. In one aspect, major changes may occur in the antigenic variants present on the surface of the agent 100 resulting in the formation of a new subtype. The appearance of new epitopes observed, for example, may occur as a result of antigenic shifts, reassortment, reshuffling, rearrangement of segments, and/or swapping of segments and generally marks the appearance of a new virulent strain of the agent 100. In one instance the prediction of the new epitopes may mark the emergence of a new strain, a new subtype, and/or the reemergence of an older strain. In this instance, natural and/or artificial humoral protection in an individual does not provide adequate protection.

Generally, when major changes do occur a larger section of the population succumbs to the infection leading to a pandemic. The problem may be alleviated in part, for example, by predicting the appearance of new strains and/or subtypes as a result of the appearance of new epitopes and/or the disappearance of existing epitopes. In one aspect, for example, including, but not limited to, the prediction of the new epitopes may be directed towards a subset of genes, for example, important for virulence and/or replication of the agent 100. For example, examining the appearance of new subtypes of influenza virus type A shows that the antigenic variations occur for the most part in the neuraminidase and/or hemagglutinin gene.

In another aspect, the selected epitope 200 may steer clear of highly variable regions and focus instead on areas of lower probability of mutations. Thus epitopes selected may circumvent hotspots of antigenic variations and target other specific regions of an agent 100, such as, for example, the receptor binding site on the surface of the agent 100. In another example, the selected epitope 200 may not be readily accessible to the immune response component, for example, the receptor binding site may be buried deep in a pocket and may be surrounded by readily accessible sequences exhibiting a higher level of antigenic variations. In this example, one possibility may include providing small antibody fragments that penetrate the receptor binding site preventing the agent 100 from binding its target. In another example, a drug and/or chemical may be used to exaggerate the accessibility of the receptor binding site. In yet another example, a chemical with a tag may be used to bind to the residue and the tag used for biding the immune response component.

In another aspect, the immune response component may be so designed so as to circumvent the shape changes in the epitope 102 and provide minimally effective binding to the epitope 102. In this example the antibody designed may include accommodations to its design by the prediction of hotspots and/or the mutational changes in the epitope 102.

In one aspect the size of the immune response component may be manipulated. For example, an immune response component, for example, the antibody 104 may be designed to include the practicably minimal binding site required to bind the epitope 102. In another example, the immune response component may be designed to the smallest effective determinant.

In one aspect, an effective treatment therapy towards a disease and/or disorder may include one or more immune response components designed to anticipate and/or treat an antigenic drift and/or an antigenic shift predicted for multiple agents. The agents need not be related to each other, for example, the therapy might be designed for an individual suffering from multiple diseases.

Following are a series of flowcharts depicting an illustrative environment for the implementation of processes. For ease of understanding, the flowcharts are organized such that the initial flowcharts present implementations via an overall “big picture” viewpoint and thereafter the following flowcharts present alternate illustrative environments and/or expansions of the “big picture” flowcharts as either sub-steps or additional steps building on one or more earlier-presented flowcharts. Those having skill in the art will appreciate that the style of presentation utilized herein (e.g., beginning with a presentation of a flowchart(s) presenting an overall view and thereafter providing additions to and/or further details in subsequent flowcharts) generally allows for a rapid and easy understanding of the various illustrative environments.

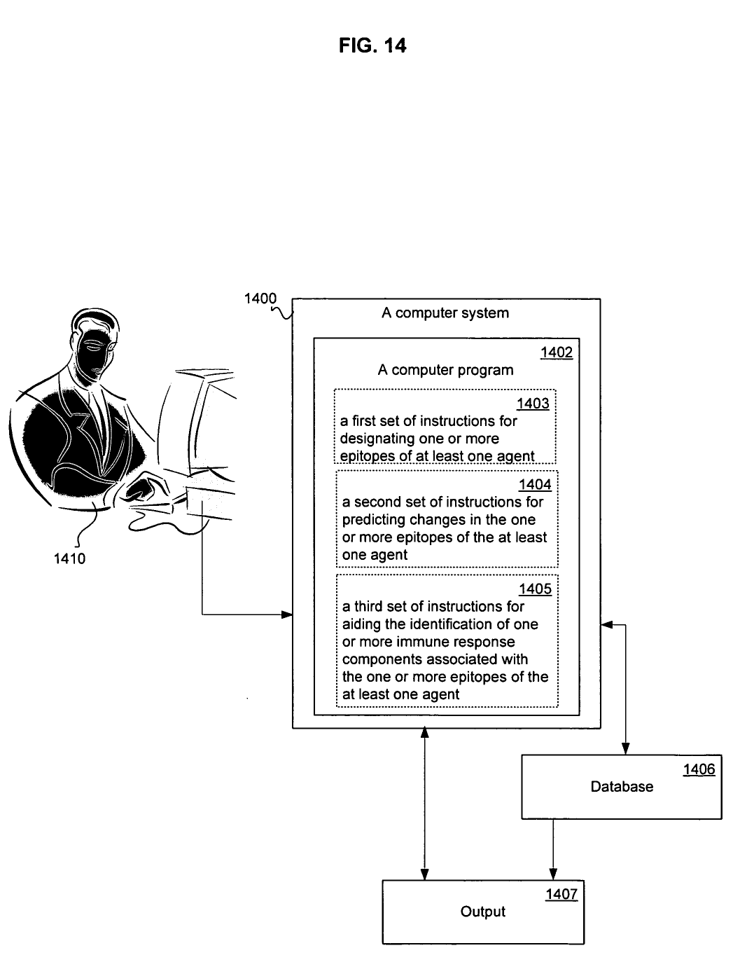

With reference now to FIG. 14, depicted is a partial view of a system that may serve as an illustrative environment of and/or for subject matter technologies. In one aspect the environment depicted includes a computer system 1400 including a computer program 1402. Depicted is the computer program 1402 including instructions 1403, 1404, and/or 1405. The computer program 1402 may include a first set of instructions for designating one or more epitopes of at least one agent 1403. The computer program 1402 may include a second set of instructions for predicting changes in the one or more epitopes of the at least one agent 1403. The computer program 1402 may include a third set of instructions for aiding the identification of one or more immune response components associated with the one or more epitopes of the at least one agent 1404. In one exemplary implementation of the system, depicted is a user 1410 (e.g., a medical professional, a researcher, a scientist, a patient, a technician, a manufacturer, a drug maker or the like) employing the system. In another exemplary implementation of the system, the computer program 1402 has access to a database 1406. In one exemplary implementation a feedback loop is set up between the computer program and the database 1406. The output 1407 may be fed back into the computer program 1402 and/or displayed on the computer system 1400. The system may be used as a research tool, as a tool for furthering treatment or the like.

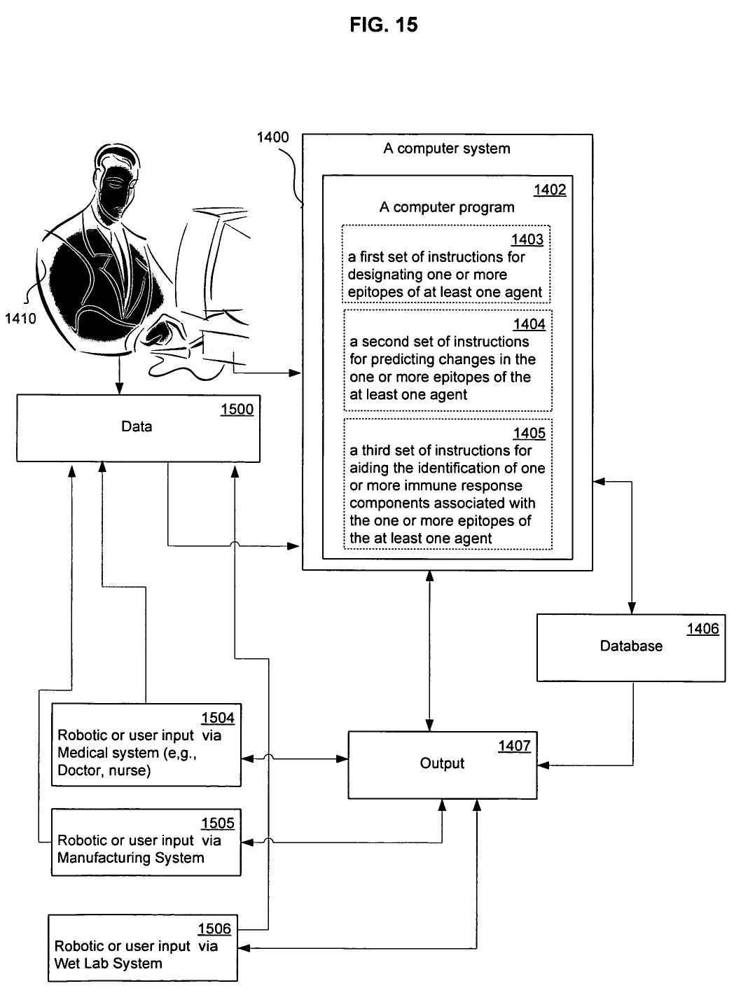

With reference now to FIG. 15, depicted is a partial view of a system that may serve as an illustrative environment of and/or for subject matter technologies. The user 1410 may input data 1500, for example, to affect the output 1407. Robotic or user input of data may also be provided via a medical system 1504, a manufacturing system 1505, or a wet lab system 1506 and the output 1407 fed back into the computer program 1402 and/or displayed on the computer system 1400.

B. Operation(s) and/or Process(es)

Following are a series of flowcharts depicting implementations of processes. For ease of understanding, the flowcharts are organized such that the initial flowcharts present implementations via an overall “big picture” viewpoint and thereafter the following flowcharts present alternate implementations and/or expansions of the “big picture” flowcharts as either sub-steps or additional steps building on one or more earlier-presented flowcharts. Those having skill in the art will appreciate that the style of presentation utilized herein (e.g., beginning with a presentation of a flowchart(s) presenting an overall view and thereafter providing additions to and/or further details in subsequent flowcharts) generally allows for a rapid and easy understanding of the various process implementations.

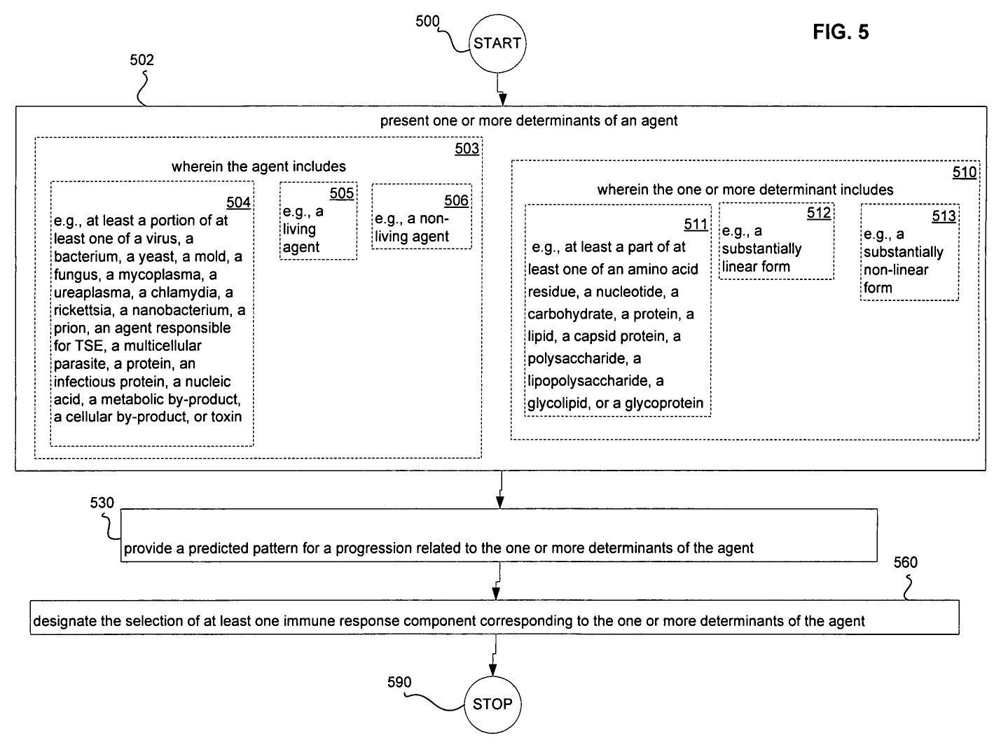

Several of the alternate process implementations are set forth herein by context. For example, as set forth herein in relation to FIG. 5, what is described as method step 504 is illustrated as a list of exemplary qualifications of an agent. Those skilled in the art will appreciate that when what is described as method step 504 is read in the context of what are described as method step 503 and method step 502, it is apparent that the list of exemplary qualifications of the agent, in context, is actually illustrative of an alternate implementation of method step 502 of presenting at least a portion of at least one of a virus, a bacterium, a yeast, a mold, a fungus, a mycoplasma, a ureaplasma, a Chlamydia, a rickettsia, a nanobacterium, a prion, an agent responsible for TSE, a multicellular parasite, a protein, an infectious protein, a nucleic acid, a metabolic by-product, a cellular by-product, or a toxin. Likewise, when what is described as method step 505 is read in the context of what are described as method step 503 and method step 502, it is apparent that, in context, method step 505 is actually illustrative of an alternate implementation of method step 502 of presenting at least a portion of a living agent. Likewise again, when what is described as method step 505 is read in the context of what are described as method step 503 and method step 502, it is apparent that, in context, method step 505 is actually illustrative of an alternate implementation of method step 502 of presenting at least a portion of a non-living agent. Contextual readings such as those just set forth in relation to method steps 504, 505, and 506 are within the ambit of one having skill in the art in light of the teaching herein, and hence are not set forth verbatim elsewhere herein for sake of clarity.

With reference now to FIG. 5, depicted are high level logic flow charts of various alternate process implementations. Method step 500 shows the start of the process. Method step 502 shows the presentation of one or more determinants. Depicted is that in various alternate implementations, method step 502 includes steps 503 and/or 510. Illustrated is that in various alternate implementations, method step 503 includes substeps 504, 505, and/or 506. Method step 503 depicts some exemplary qualifications of an agent. As depicted method step 504 may include at least a portion of at least one of a virus, a bacterium, a yeast, a mold, a fungus, a mycoplasma, a ureaplasma, a Chlamydia, a rickettsia, a nanobacterium, a prion, an agent responsible for TSE, a multicellular parasite, a protein, an infectious protein, a nucleic acid, a metabolic by-product, a cellular by-product, and/or a toxin. The agent may include a living agent method step 504 and/or a non-living agent 506 of an agent. Method step 510 depicts the one or more determinants and includes additional steps 511, 512, and/or 513. Method step 511 depicts including the one or more determinants wherein the one or more determinants include at least a part of at least one of an amino acid residue, a nucleotide, a carbohydrate, a protein, a lipid, a capsid protein, a polysaccharide, a lipopolysaccharide, a glycolipid, or a glycoprotein. Method step 512 depicts wherein the one or more determinants may include substantially linear determinants. Method step 513 depicts wherein the one or more determinants may include non-linear determinants. It will also be appreciated by those skilled in the art that method step 500 may include accepting input related to, for example, the agent, the one or more determinants and/or other relevant criteria such as a size of the determinant, a type of the determinant, a nature of the disease, a disorder and/or a condition requiring management, and/or a sensitivity of a group requiring management. Method step 530 depicts providing a predicted pattern for the progression related to the one or more determinants of the agent. For example, previous pattern changes known and/or predicted may be used to extrapolate future progressions of the pattern changes that may be observed in the one or more determinants of the agent. Method step 560 depicts designating the selection of at least one immune response component corresponding to the one or more determinants of the agent. The immune response components so designated may include those for managing a disease, a condition or for managing a response, for example. Method step 590 shows the end of the process.

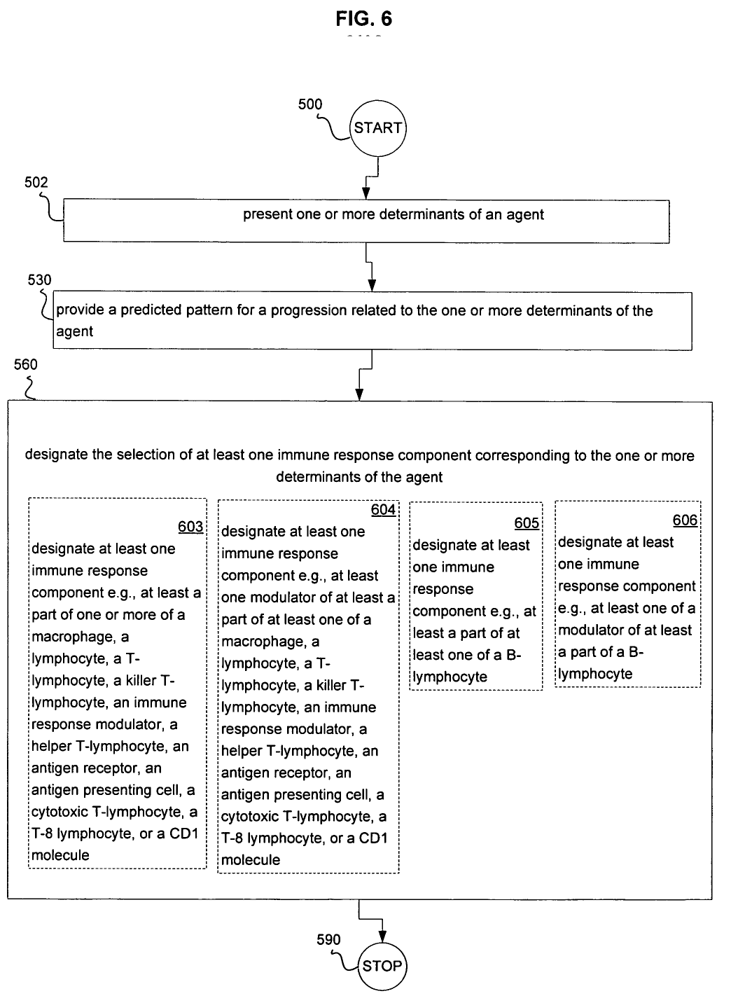

With reference now to FIG. 6, depicted is a high-level logic flowchart depicting alternate implementations of the high-level logic flowchart of FIG. 5. Depicted is that method step 560 includes method step 603, 604, 605, and/or 606. Method step 603 depicts designating at least one immune response component, such as, for example, including but not limited to, of at least a part of one or more of a macrophage, a lymphocyte, a T-lymphocyte, a killer T-lymphocyte, an immune response modulator, a helper T-lymphocyte, an antigen receptor, an antigen presenting cell, a cytotoxic T-lymphocyte, a T-8 lymphocyte, or a cluster differentiation molecule such as a CD3 and/or a CD1 molecule. Method step 604 shows designating at least one immune response component, such as, for example, including but not limited to, at least one modulator of at least a part of at least one of a macrophage, lymphocyte, a T-lymphocyte, a killer T-lymphocyte, an immune response modulator, a helper T-lymphocyte, an antigen receptor, an antigen presenting cell, a cytotoxic T-lymphocyte, a T-8 lymphocyte, a cluster differentiation molecule, a CD3 molecule and/or a CD1 molecule. Method step 605 shows designating at least one immune response component, such as, for example, at least a part of at least one of a B-lymphocyte. Method step 606 shows designating at least one immune response component, for example, at least one of a modulator of at least a part of a B-lymphocyte.

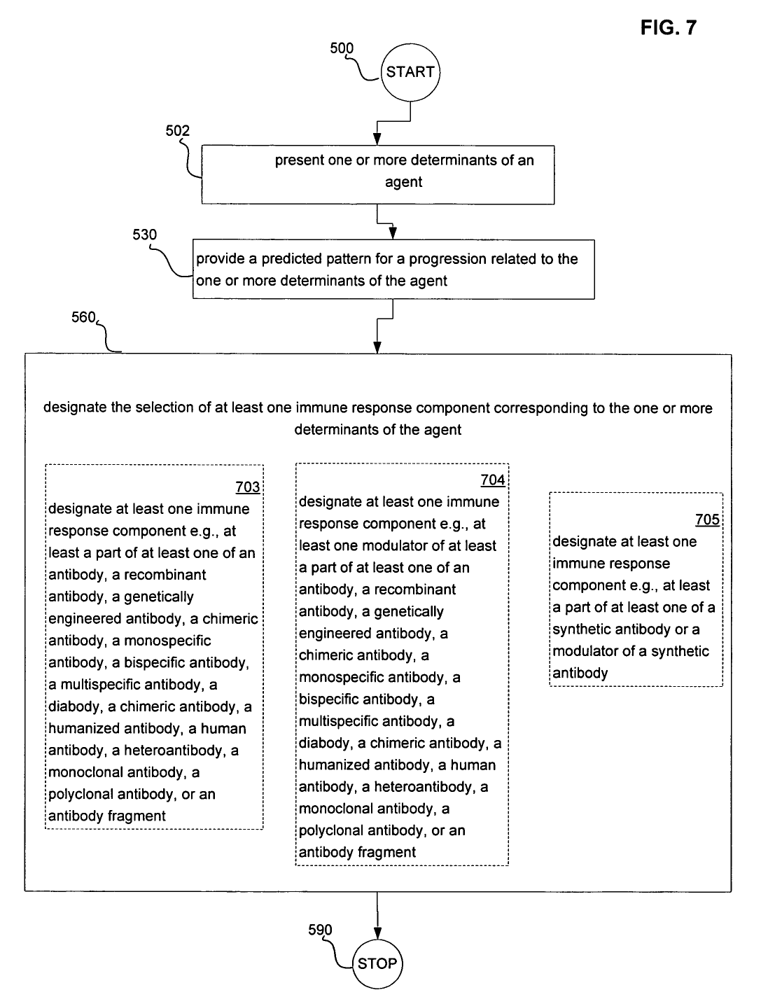

Referring now to FIG. 7, depicted is a high-level logic flowchart depicting alternate implementations of the high-level logic flowchart of FIG. 5. Depicted is that in various alternate implementations method step 560 includes method step 703, 704, and/or 705. Method step 703 shows designating at least one immune response component, for example, at least a part of at least one of an antibody, a recombinant antibody, a genetically engineered antibody, a chimeric antibody, a monospecific antibody, a bispecific antibody, a multispecific antibody, a diabody, a chimeric antibody, a humanized antibody, a human antibody, a heteroantibody, a monoclonal antibody, a polyclonal antibody, and/or an antibody fragment. Method step 704 depicts designating at least one immune response component, for example, at least one modulator of at least a part of at least one of an antibody, a recombinant antibody, a genetically engineered antibody, a chimeric antibody, a monospecific antibody, a bispecific antibody, a multispecific antibody, a diabody, a chimeric antibody, a humanized antibody, a human antibody, a heteroantibody, a monoclonal antibody, polyclonal antibody, and/or an antibody fragment. Method step 705 illustrates designating at least one immune response component e.g., at least a part of at least one of a synthetic antibody or a modulator of a synthetic antibody.

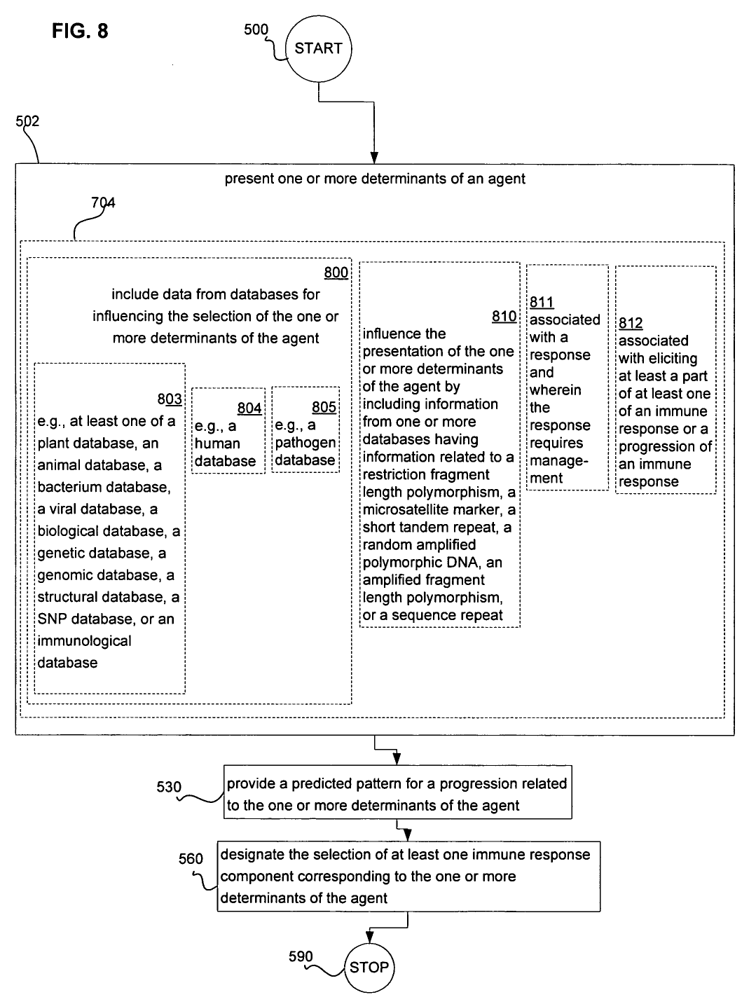

Referring now to FIG. 8, depicted is a high-level logic flowchart depicting alternate implementations of the high-level logic flowchart of FIG. 5. In one alternate implementation, as depicted in FIG. 8, method step 502 includes method steps 800, 810, 811, and/or 812. Method step 800 depicts including data from databases for influencing the selection of the one or more determinants of the agent. Method step 800 also includes additional steps 803, 804 and/or 805. Method step 803 depicts including at least one of a plant database, an animal database, a bacterium database, a viral database, a biological database, a genetic database, a genomic database, a structural database, a SNP database, or an immunological database. Method step 804 and 805 depicts including a human database or a pathogen database, respectively, for influencing the selection of the one or more determinants.

Continuing to refer to FIG. 8, method step 810 shows influencing the presentation of the one or more determinants of the agent by including information from one or more databases having information related to a restriction fragment length polymorphism, a microsatellite marker, a short tandem repeat, a random amplified polymorphic DNA, an amplified fragment length polymorphism, or a sequence repeat. Method step 811 depicts presenting one or more determinants of an agent associating with a response and wherein the response requires management (e.g., a biological response). Method step 812 depicts presenting one or more determinants of an agent associated with eliciting at least a part of at least one of an immune response or a progression of an immune response.

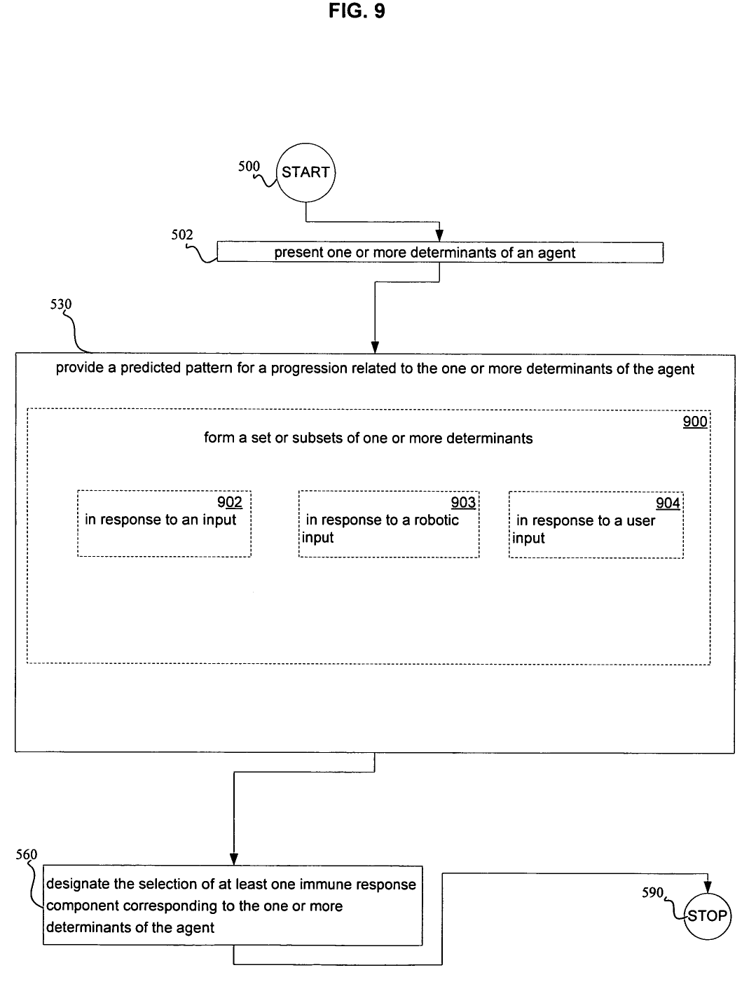

With reference now to FIG. 9, depicted is a high-level logic flowchart depicting alternate implementations of the high-level logic flowchart of FIG. 5. In one alternate implementation, as depicted in FIG. 9, method step 530 includes method steps 900. Method step 900 depicts forming a set or a subset (e.g., a group of one or more determinants). The set or subset may be formed in response to an input method step 902 (e.g., biological criteria, geographical criteria or other substantive criteria), in response to a robotic input method step 903 and/or in response to a user input method step 904.

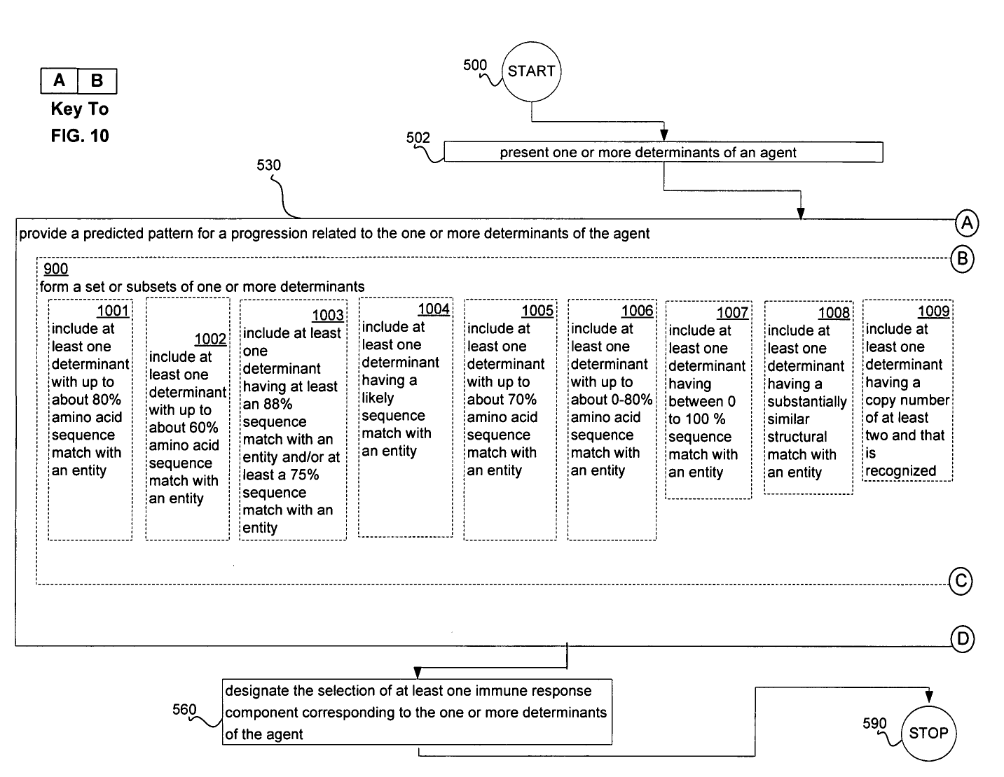

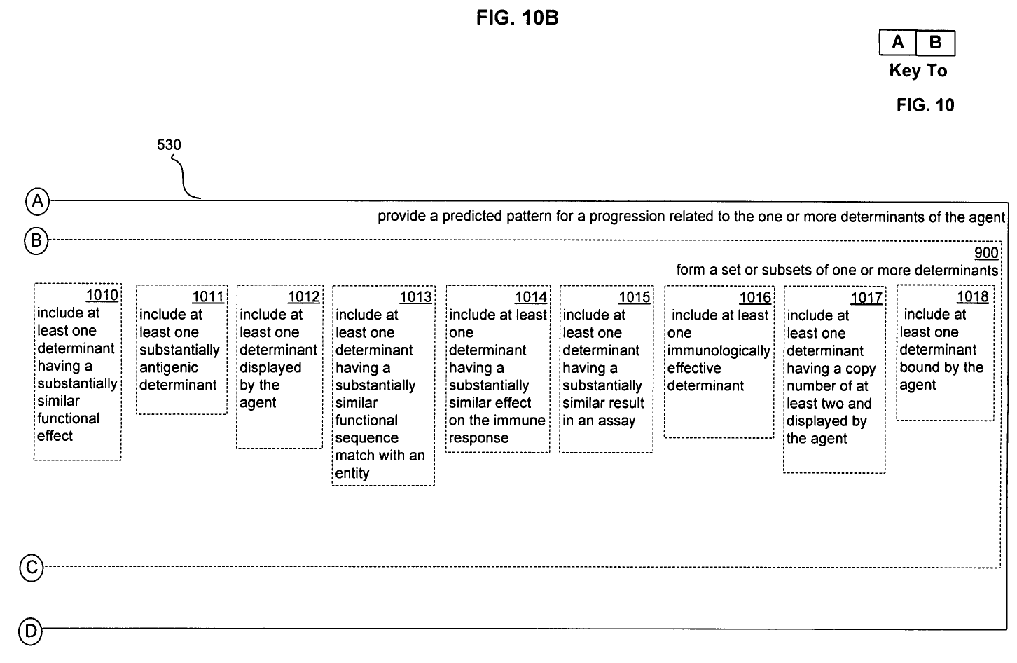

With reference now to FIG. 10, depicted is a high-level logic flowchart depicting alternate implementations of the high-level logic flowchart of FIG. 5. Shown is one alternate implementation, method step 530 includes method steps 1000-1018. The criteria used to form sets or subsets may include at least one determinant with up to about 80% amino acid sequence match with an entity method step 1001. Method step 1002 depicts forming set or subsets by including at least one determinant with up to about 60% amino acid sequence match with an entity. Method step 1003 depicts forming set or subsets by including at least one determinant having at least 88% sequence match with an entity and/or at least a 75% sequence match with an entity. Method step 1004 depicts forming set or subsets by including at least one determinant having a likely sequence match with an entity. Method step 1005 depicts forming set or subsets by including at least one determinant with up to about 70% amino acid sequence match with an entity. Method step 1006 depicts forming set or subsets by including at least one determinant with up to about 0-80% amino acid sequence match with an entity. Method step 1007 depicts forming set or subsets by including at least one determinant having between 0 to 100% sequence match with an entity. Method step 1008 depicts forming set or subsets by including at least one determinant having a substantially similar structural match with an entity. Method step 1009 depicts forming set or subsets by including at least one determinant having a copy number of at least two and that is recognized (e.g., by the occurrence of an immune response directed towards the one or more determinants).

Continuing to refer to FIG. 10, method step 1010 depicts forming set or subsets by including at least one determinant having a substantially similar functional effect. Method step 1011 depicts forming set or subsets by including at least one substantially antigenic determinant. Method step 1012 depicts forming set or subsets by including at least one determinant displayed by the agent (e.g., on the surface of the agent). Method step 1013 depicts forming set or subsets by including at least one determinant having a substantially similar functional sequence match with an entity. Method step 1014 depicts forming set or subsets by including at least one determinant having a substantially similar effect on the immune response. Method step 1015 depicts forming set or subsets by including at least one determinant having a substantially similar result in an assay. Method step 1016 depicts forming set or subsets by including at least one immunologically effective determinant. Method step 1017 depicts forming set or subsets by including at least one determinant having a copy number of at least two and displayed by the agent. Method step 1018 depicts forming set or subsets by including at least one determinant bound by the agent (e.g., a cofactor, or an ectopic determinant that may be part of the agent or not part of the agent).

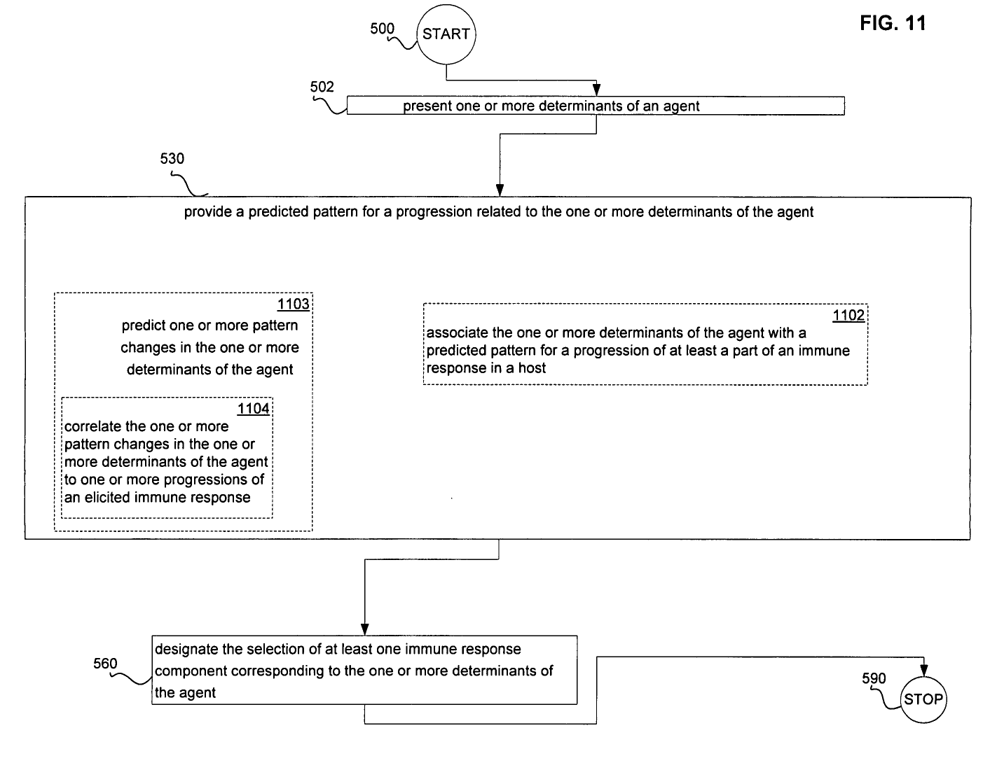

With reference now to FIG. 11, depicted is a high-level logic flowchart depicting alternate implementations of the high-level logic flowchart of FIG. 5. Depicted is that method step 530 includes method step 1102 and/or 1103. Method step 1102 shows associating the one or more determinants of the agent with a predicted pattern for a progression of at least a part of an immune response in a host. Method step 1103 shows predicting one or more pattern changes in the one or more determinants of the agent. Method step 1103 includes method step 1104 which depicts correlating the one or more pattern changes in the one or more determinants of the agent to one or more progressions of an elicited immune response.

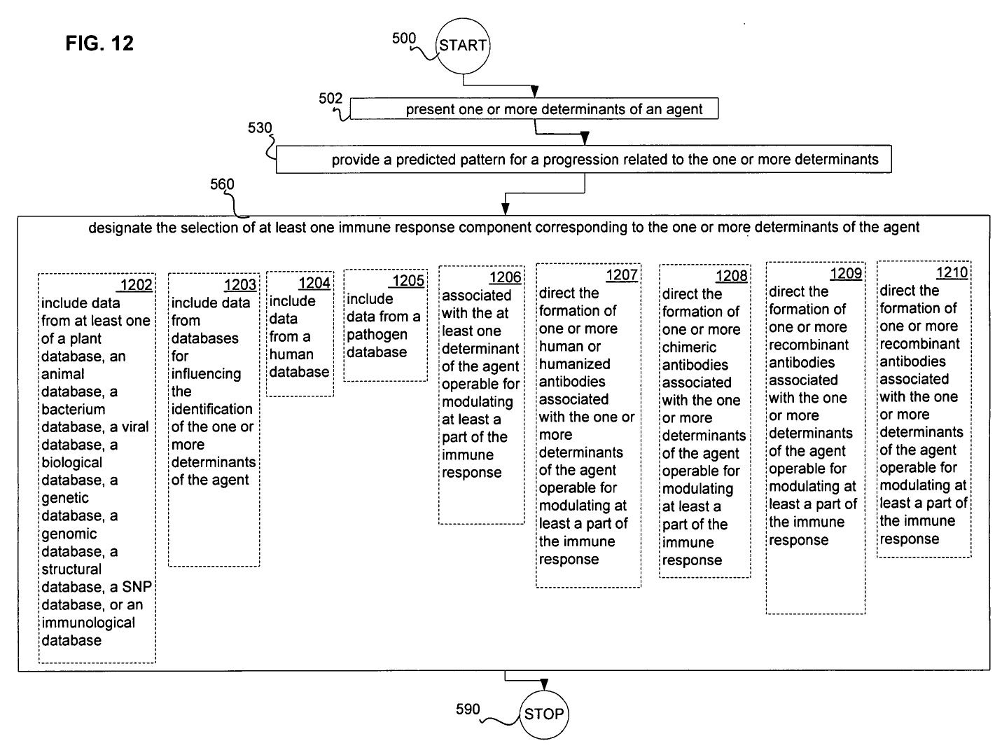

Referring now to FIG. 12, depicted is a high-level logic flowchart depicting alternate implementations of the high-level logic flowchart of FIG. 5. Depicted is that method step 560 includes method step 1202-1210. Method step 1202 shows including data from at least one of a plant database, an animal database, a bacterium database, a viral database, a biological database, a genetic database, a genomic database, a structural database, a SNP database, or an immunological database. Method step 1203 shows including data from databases for influencing the identification of the one or more determinants of the agent. Method step 1204 shows including data from a human database. Method step 1205 shows including data from a pathogen database. Method step 1206 shows including designating the selection of at least one immune response component corresponding to the one or more determinants of the agent associated with the at least one determinant of the agent operable for modulating at least a part of the immune response. Method step 1207 shows including directing the formation of one or more human or humanized antibodies associated with the one or more determinants of the agent operable for modulating at least a part of the immune response. Method step 1208 shows including directing the formation of one or more chimeric antibodies associated with the one or more determinants of the agent operable for modulating at least a part of the immune response. Method step 1209 shows including directing the formation of one or more recombinant antibodies associated with the one or more determinants of the agent operable for modulating at least a part of the immune response. Method step 1210 shows including directing the formation of one or more recombinant antibodies associated with the one or more determinants of the agent operable for modulating at least a part of the immune response.

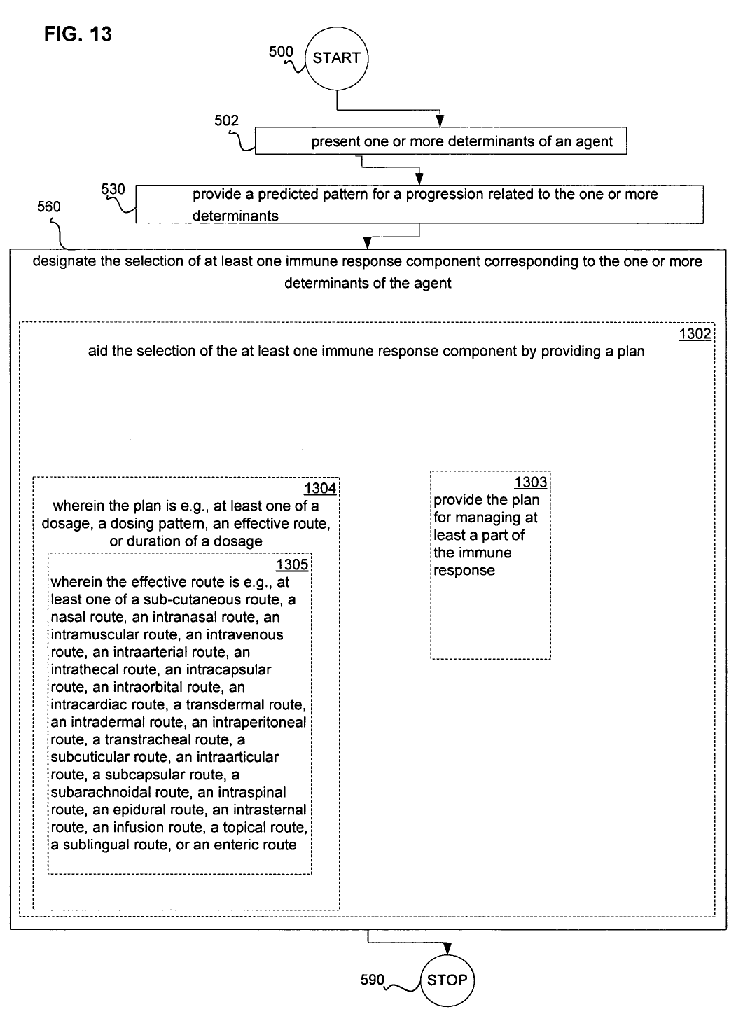

With reference now to FIG. 13, depicted is a high-level logic flowchart depicting alternate implementations of the high-level logic flowchart of FIG. 5. Depicted is that method step 560 includes method step 1302. Method step 1302 includes aiding the selection of the at least one immune response component by providing a plan (e.g., a scheme, a list of options, or a course of action). What is shown is that method step 1302 includes additional method step 1303 and/or 1304. Method step 1303 includes providing the plan for managing at least a part of the immune response. Method step 1304 includes wherein the plan is e.g., at least one of a dosage, a dosing pattern, an effective route, or duration of a dosage. Method step 1304 includes additional method step 1305 wherein the effective route is e.g., at least one of a sub-cutaneous route, a nasal route, an intranasal route, an intramuscular route, an intravenous route, an intraarterial route, an intrathecal route, an intracapsular route, an intraorbital route, an intracardiac route, a transdermal route, an intradermal route, an intraperitoneal route, a transtracheal route, a subcuticular route, an intraarticular route, a subcapsular route, a subarachnoidal route, an intraspinal route, an epidural route, an intrasternal route, an infusion route, a topical route, a sublingual route, or an enteric route.

### C. Variation(s), and/or Implementation(s)

Those having skill in the art will recognize that the present application teaches modifications of the devices, structures, and/or processes within the spirit of the teaching herein. For example, in one aspect, the immune response components may be formulated to cross the blood-brain barrier which is known to exclude mostly hydrophilic compounds. For example, an antibody fragment may be encased in a lipid vesicle. In another example, the antibody or a portion of the antibody may be tagged onto a carrier protein or molecule. In another example, an antibody may be split into one or more complementary fragments, each fragment encased by a lipid vesicle, and each fragment functional only on binding its complementary fragment. Once the blood-brain barrier has been crossed the lipid vesicle may be dissolved to release the antibody fragments which reunite with their complementary counterparts and form a fully functional antibody. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect the immune response components may be made in large format. The method lends itself to both small format or personalized care applications and large scale applications. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, the method may be used to designate immune response components for any diseases or disorders. The application of this method is not limited to diseases where antigenic shift or drift keeps the immune system guessing making it slow to respond. Although, influenza or aids are likely candidates other diseases, disorders and/or conditions will likely benefit from this methodology. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, real-time evaluation may be provided of the antigenic changes by including a portable PCR machine which samples the environment for strains locally present. The information may be sent remotely to another location or to a portable drip patch utilized by the person resulting in the activation of the necessary immune response components providing adequate protection. As the evaluation changes the portable drip patch may be triggered to change the dosage or type of immune response component delivered. Such a portable drip patch operably-coupled to a portable PCR machine has wide variety of application, for example, including, but not limited to, when medical personnel visit areas endemic to a disease, or when military personnel visit hostile territory. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, an individual may use a drip-patch infused with the immune response components preprogrammed to provide the user the necessary protection over a period of time, and to anticipate pattern changes in the epitopes of the agent 100. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, RNA blockers, or single stranded RNAI technology may be used to downregulate genes or components of the immune system in conjunction with the method. Other modifications of the subject matter herein will be appreciated by one of skill in the art in light of the teachings herein.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter subject matter described herein applies equally regardless of the particular type of signal bearing media used to actually carry out the distribution. Examples of a signal bearing media include, but are not limited to, the following: recordable type media such as floppy disks, hard disk drives, CD ROMs, digital tape, and computer memory; and transmission type media such as digital and analog communication links using TDM or IP based communication links (e.g., packet links).

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment).

Those skilled in the art will recognize that it is common within the art to describe devices and/or processes in the fashion set forth herein, and thereafter use standard engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing-position and/or velocity; control motors for moving and/or adjusting components and/or quantities). A typical data processing system may be implemented utilizing any suitable commercially available components, such as those typically found in data computing/communication and/or network computing/communication systems.

All of the referenced U.S. patents, U.S. patent application publications, U.S. patent applications, foreign patents, foreign patent applications, and/or non-patent publications referred to in this specification and/or listed in any Application Data Sheet, are incorporated herein by reference, in their entireties.

The herein described aspects depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components.

While particular aspects of the present subject matter described herein have been shown and described, it will be apparent to those skilled in the art that, based upon the teachings herein, changes and modifications may be made without departing from this subject matter described herein and its broader aspects and, therefore, the appended claims are to encompass within their scope all such changes and modifications as are within the true spirit and scope of this subject matter described herein. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.).

1. A method, comprising:
delivering a treatment to a host wherein the treatment is associated with modulating a predicted pattern of progression of one or more antigenic determinants.

2. The method of claim 1, wherein the treatment further comprises:
delivering at least a part of one or more of a macrophage, a lymphocyte, a T-lymphocyte, a killer T-lymphocyte, an immune response modulator, a helper T-lymphocyte, an antigen receptor, an antigen presenting cell, a cytotoxic T-lymphocyte, a T-8 lymphocyte, a cluster differentiation (CD) molecule, a CD3 molecule, or a CD1 molecule.

3. The method of claim 1, wherein the treatment further comprises:
delivering one or more modulators of at least a part of at least one of a macrophage, a lymphocyte, a T-lymphocyte, a killer T-lymphocyte, an immune response modulator, a helper T-lymphocyte, an antigen receptor, an antigen presenting cell, a cytotoxic T-lymphocyte, a T-8 lymphocyte, a cluster differentiation (CD) molecule, a CD3 molecule, or a CD1 molecule.

4. The method of claim 1, wherein the treatment further comprises:
delivering at least a part of a B-lymphocyte.

5. The method of claim 1, wherein the treatment further comprises:
delivering at least one of a modulator of at least a part of a B-lymphocyte.

6. The method of claim 1, wherein the treatment further comprises:
delivering at least a part of one or more of an antibody, a recombinant antibody, a genetically engineered antibody, a chimeric antibody, a monospecific antibody, a bispecific antibody, a multispecific antibody, a diabody, a chimeric antibody, a humanized antibody, a human antibody, a heteroantibody, a monoclonal antibody, a polyclonal antibody, or an antibody fragment.

7. The method of claim 1, wherein the treatment further comprises:
delivering one or more modulators of at least a part of at least one of an antibody, a recombinant antibody, a genetically engineered antibody, a chimeric antibody, a monospecific antibody, a bispecific antibody, a multispecific antibody, a diabody, a chimeric antibody, a humanized antibody, a human antibody, a heteroantibody, a monoclonal antibody, a polyclonal antibody, or an antibody fragment.

8. The method of claim 1, wherein the delivering a treatment to a host further comprises:
delivering the treatment by at least one of a sub-cutaneous route, a nasal route, an intranasal route, an intramuscular route, an intravenous route, an intraarterial route, an intrathecal route, an intracapsular route, an intraorbital route, an intracardiac route, a transdermal route, an intradermal route, an intraperitoneal route, a transtracheal route, a subcuticular route, an intraarticular route, a subcapsular route, a subarachnoidal route, an intraspinal route, an epidural route, an intrasternal route, an infusion route, a topical route, a sublingual route, or an enteric route.

9. A system comprising, comprising:
circuitry for suggesting delivery of a treatment to a host wherein the treatment is associated with modulating a predicted pattern of progression of one or more antigenic determinants.

10. The system of claim 9, wherein the treatment further comprises:
delivering at least a part of one or more of a macrophage, a lymphocyte, a T-lymphocyte, a killer T-lymphocyte, an immune response modulator, a helper T-lymphocyte, an antigen receptor, an antigen presenting cell, a cytotoxic T-lymphocyte, a T-8 lymphocyte, a cluster differentiation (CD) molecule, a CD3 molecule, or a CD1 molecule.

11. The system of claim 9, wherein the treatment further comprises:
delivering one or more modulators of at least a part of at least one of a macrophage, a lymphocyte, a T-lymphocyte, a killer T-lymphocyte, an immune response modulator, a helper T-lymphocyte, an antigen receptor, an antigen presenting cell, a cytotoxic T-lymphocyte, a T-8 lymphocyte, a cluster differentiation (CD) molecule, a CD3 molecule, or a CD1 molecule.

12. The system of claim 9, wherein the treatment further comprises:
delivering at least a part of a B-lymphocyte.

13. The system of claim 9, wherein the treatment further comprises:
delivering at least one of a modulator of at least a part of a B-lymphocyte.

14. The system of claim 9, wherein the treatment further comprises:
delivering at least a part of one or more of an antibody, a recombinant antibody, a genetically engineered antibody, a chimeric antibody, a monospecific antibody, a bispecific antibody, a multispecific antibody, a diabody, a chimeric antibody, a humanized antibody, a human antibody, a heteroantibody, a monoclonal antibody, a polyclonal antibody, or an antibody fragment.

15. The system of claim 9, wherein the treatment further comprises:
delivering one or more modulators of at least a part of at least one of an antibody, a recombinant antibody, a genetically engineered antibody, a chimeric antibody, a monospecific antibody, a bispecific antibody, a multispecific antibody, a diabody, a chimeric antibody, a humanized antibody, a human antibody, a heteroantibody, a monoclonal antibody, a polyclonal antibody, or an antibody fragment.

16. The system of claim 9, wherein the delivering a treatment to a host further comprises:
delivering the treatment by at least one of a sub-cutaneous route, a nasal route, an intranasal route, an intramuscular route, an intravenous route, an intraarterial route, an intrathecal route, an intracapsular route, an intraorbital route, an intracardiac route, a transdermal route, an intradermal route, an intraperitoneal route, a transtracheal route, a subcuticular route, an intraarticular route, a subcapsular route, a subarachnoidal route, an intraspinal route, an epidural route, an intrastemal route, an infusion route, a topical route, a sublingual route, or an enteric route.

17. A system, comprising:
a computer readable medium including, but not limited to, a computer program for use with a computer system and wherein the computer program includes a plurality of instructions including:
one or more instructions for selecting one or more determining elements;
one or more instructions for predicting at least one pattern change in the one or more determining elements;
one or more instructions for associating the at least one pattern change in the one or more determining elements with at least one outcome; and
one or more instructions for designating a course of action associated with the at least one pattern change in the one or more determining elements.

18. The system of claim 17, wherein the computer readable medium further comprises:
one or more instructions for identifying one or more treatments corresponding to the one or more determining elements.

19. The system of claim 17, wherein the computer readable medium further comprises:
one or more instructions for identifying one or more treatments responsive for modulating a host defense mechanism.

20. The system of claim 17, wherein the one or more instructions for designating a course of action further comprises:
one or more instructions for including at least one parameter including a location, number, an antigenicity profile, a type of immune response component, an availability of an antibody, a sequence match with a host, or a sequence match with an agent.

21. The system of claim 17, wherein the at least one outcome further comprises:
an outcome predicting a pattern of change in one or more immune response components.

22. A system, comprising:
circuitry for selecting one or more determining elements;
circuitry for predicting at least one pattern change in the one or more determining elements;
circuitry for associating the at least one pattern change in the one or more determining elements with at least one outcome; and
circuitry for designating a course of action associated with the at least one pattern change in the one or more determining elements.

23. A system, comprising:
means for selecting one or more determining elements;
means for predicting at least one pattern change in the one or more determining elements;
means for associating the at least one pattern change in the one or more determining elements with at least one outcome; and
means for designating a course of action associated with the at least one pattern change in the one or more determining elements.
